(19) United States
(12) Patent Application Publication
(10) Pub. No.: US 2002/0115968 A1
(43) Pub. Date: Aug. 22, 2002

(54) Absorbing article for skin care and antibiotic

(76) Inventor: Shih-chiang Lin, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/785,281

(22) Filed: Feb. 20, 2001

(51) Int. Cl. A61F 13/15; A61F 13/20

(52) U.S. Cl. 604/360; 604/374; 604/375; 604/385.06

(57) ABSTRACT

An absorbing article for skin care and antisepsis comprises a substrate having absorbing ability for liquid and powder, and an augmentation layer made of chitosan and added to the substrate. The augmentation layer is made of powder, particle, liquid or solution of chitosan, and added to surface or inner part of the substrate, thus forming a protective layer on the substrate. The protective layer has natural biological care to skin, and has deodorization, antibiotic and sterilization effect, thus preventing bedsore.

### FIELD OF THE INVENTION

The present invention relates to an absorbing article for skin care and antisepsis, especially to a substrate with chitosan layer, wherein the substrate can be used as absorbing article for protecting skin.

### BACKGROUND OF THE INVENTION

The conventional diapers or sanitary napkins are made of unwoven cloth, which are stifling after long time use. The user may have the problem of bedsore, heat rash or tainted and will be very uneasy. To solve this problem the unwoven cloth is coated with natural or artificial materials such as mineral jelly or aloe, or waterless lubricant. However, the surface of the unwoven cloth will be hardened and the friction is increased to result more unease. Moreover, in above treatment for unwoven cloth, the unwoven cloth is treated by chemical and certain harmful material such as P is remained. The residual phosphorus is harmful to human after long-term use.

### SUMMARY OF THE INVENTION

It is the object of the present invention to provide an absorbing article for skin care and antibiotic. The material used for the absorbing article should block the natural organism growing on the cloth and be lubricative to the skin. The substrate is mainly composed of cloth with a chitosan augmentation layer. The chitosan is a natural high molecular number chitin abstracted from shell of shrimp and crab or bone of cuttle, and is processed by deacetylation step. The degree of deacetylation is generally 65-99% and, most often, 70-90%. The chitosan augmentation layer forms a protective layer with natural biological care to skin, and has deodorization, antibiotic and sterilization effect, thus preventing bedsore.

The absorbing article for skin care and antibiotic according to the present invention has a chitosan augmentation layer provided on inner and outer side of a substrate, thus forming a protective layer on the substrate. The substrate can be of cotton, cloth or paper materials with absorbing ability. For example, the substrate could be sanitary napkin, nappy, pet pad, etc.

The various objects and advantages of the present invention will be more readily understood from the following detailed description when read in conjunction with the appended drawing, in which:

### BRIEF DESCRIPTION OF DRAWING

FIG. 1 is the perspective view of the present invention;

FIG. 2 is the exploded view of the present invention;

FIG. 3 is a perspective view showing the manufacture of the present invention;

FIG. 4 showing the chitosan is applied to a plurality layer for the substrate; and

FIG. 5 shows another preferred embodiment of the present invention.

### DETAILED DESCRIPTION OF THE INVENTION

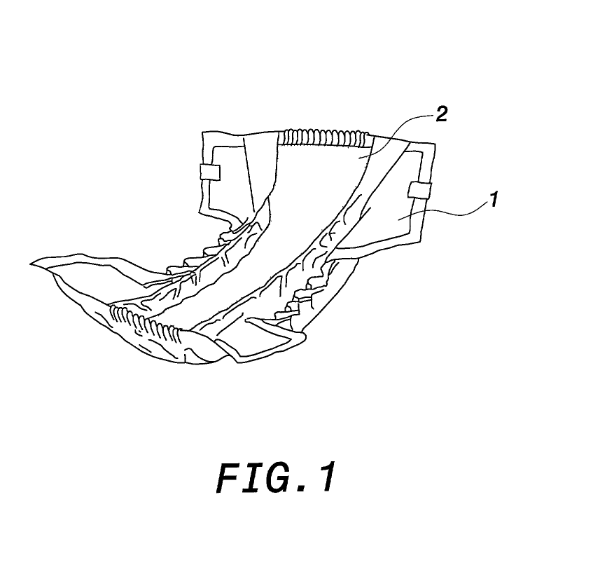

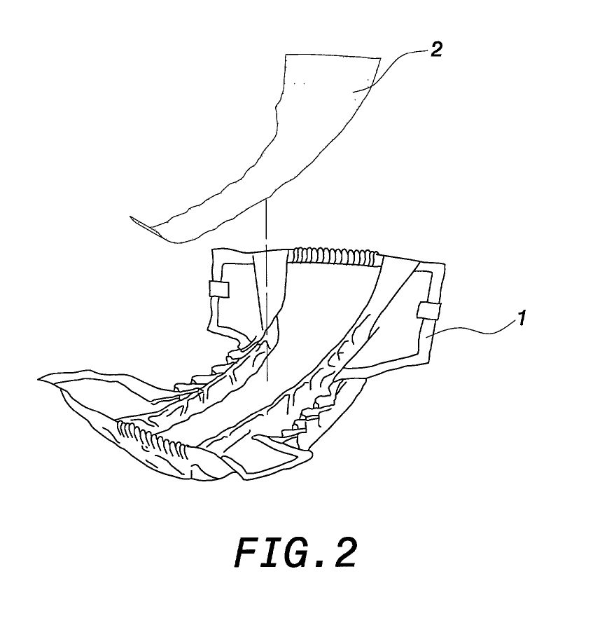

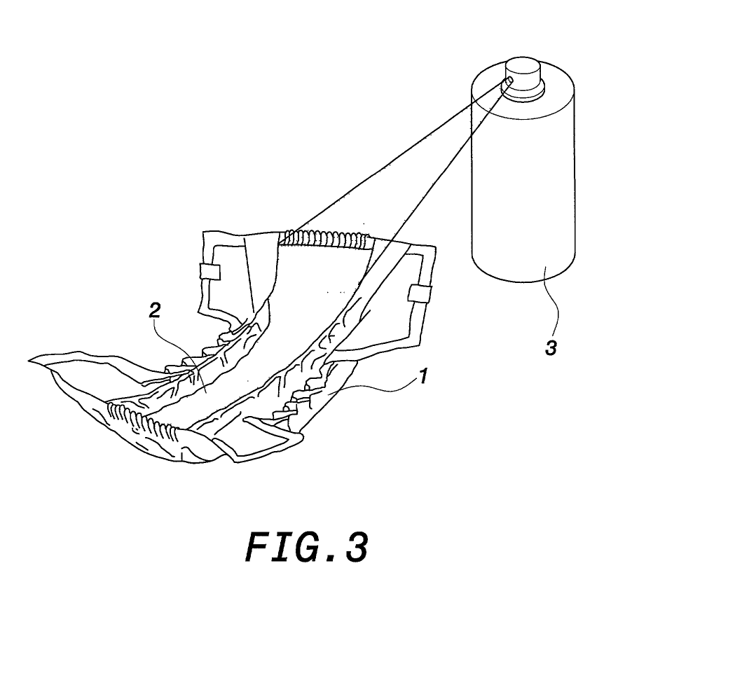

With reference now to FIGS. 1 to 4, the present invention provides an absorbing article for skin care and antibiotic, which comprises a substrate 1 and an augmentation layer 2. The substrate is an absorbing body, which can absorb liquid or powder. In this case, the absorbing body absorbs liquid such as urine or menses secretion. For example, the absorbing body can be sanitary napkin or nappy, which can absorb large amount of liquid or powder on the surface thereof. In the case that the powder is egestion or dirt, the absorbing body can be nursing pad or pet pad.

The substrate 1 is attached with the augmentation layer 2, which is made chitosan. The augmentation layer 2 can be powder, particle or solution containing chitosan and applied on surface or inner side of the substrate 1. Alternatively, the augmentation layer 2 can be solution containing chitosan, or liquid containing chitosan and mixed into solution, thus forming a protective film on the substrate 1. Firstly, the chitosan powder is mixed into weak acid solution wherein the pH value is controlled within 1.0-7.5 and the amount of the chitosan powder is 0.05%-15%.

When the chitosan is in the form of liquid or solution, the chitosan can be applied to the substrate 1 by spraying, coating, brushing, dipping or dying such that the substrate 1 contains the chitosan. In case that the chitosan is in the form of powder, the chitosan powder can be applied to surface or inner side of the substrate 1 by spraying, embedding, attaching or binding. The substrate 1 is an absorbing body; therefore, the substrate 1 can absorb the chitosan powder on surface thereof

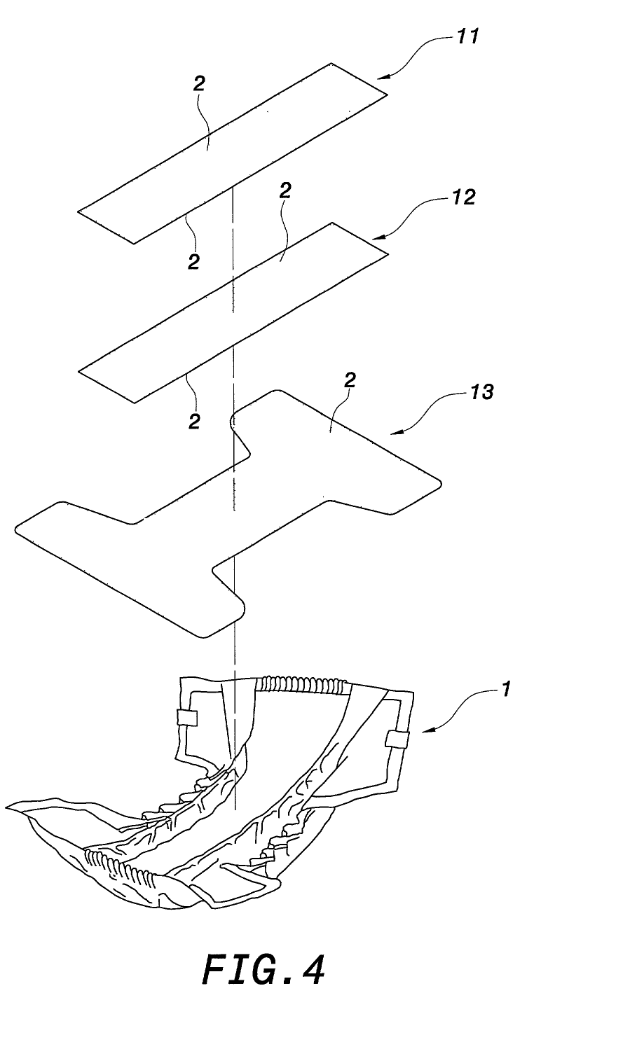

FIG. 4 shows a preferred embodiment of the present invention wherein the substrate 1 is a nappy and only the portion of the substrate 1 applied with augmentation layer is demonstrated. The portion of the substrate 1 applied with augmentation layer comprises an upper cloth layer 11, a guiding and absorbing layer 12 and a kernel absorbing layer 13. The upper cloth layer 11 and the guiding and absorbing layer 12 are applied with chitosan on the outer and inner surfaces thereof, while the kernel absorbing layer 13 is applied with chitosan on the outer surfaces thereof only. In this preferred embodiment, the kernel absorbing layer 13 comprises a cotton layer on outer side and a reinforcing cotton layer on inner side. The guiding and absorbing layer 12 is arranged on outer side of the kernel absorbing layer 13; and the upper cloth layer 11 is arranged on outer side of guiding and absorbing layer 12. The upper cloth layer 11 and the guiding and absorbing layer 12 are made of unwoven cloth. After those layers are arranged on the substrate 1, the chitosan solution or powder is applied to the substrate by a nozzle 3. Afterward, the substrate 1 is naturally dried or dried by a drying process.

The chitosan augmentation layer provides a protective layer on the substrate 1 and cares the skin in a moderate and biological way. The chitosan augmentation layer provides deodorization, antibiotic and sterilization effect and prevents bedsore.

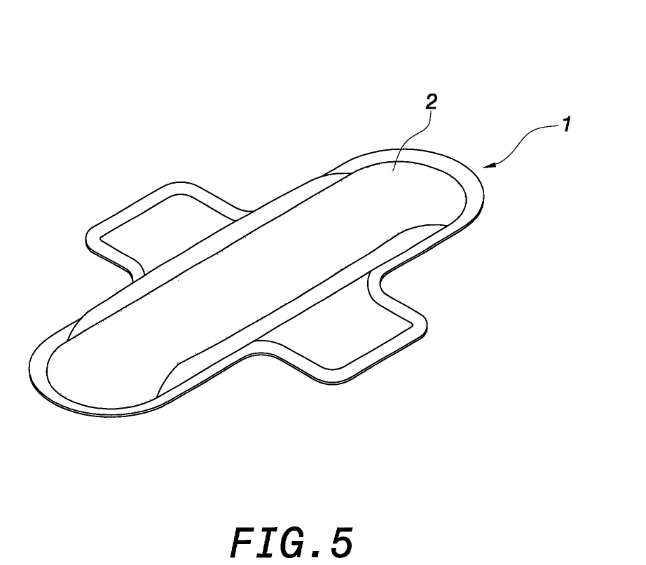

FIG. 5 shows another preferred embodiment of the present invention wherein the substrate 1 is a sanitary napkin.

The chitin is a natural polymer and taken from shell of crustacean animal such as shrimp and crab; and from the cell wall of mushroom. The chitosan is the chitin subjected to deacetylation reaction.

In recent years, scientists found that the chitosan has excellent medical function including compatible to human body composition, biological decomposition, antibiotic, facilitating coalescence, suppressing cancer growth. Therefore, the chitosan has been applied to operation suture, artificial skin and medicine, which are nontoxic and eatable.

Alternatively, in the present invention, the chitosan is solved in a weak-acid solution and then the resulting solution is sprayed to a cloth substrate to form a protective layer and have above-mentioned excellent qualities after in contact with human skin.

The acid environment facilitates to suppress growth of germ. The chitosan has various content according to the radical thereof. In the present invention, for example, the chitosan is added to a nappy mainly by spraying such that the chitosan is about 1-200 g/m² per nappy. The weight of the chitosan is about 0.01-3% of the absorbing article, which is little amount for the nappy. There is left about 97% space for absorbing in the nappy. The chitosan does not need a drying process and a drying process can be carried by lamp, ultraviolet, or drier, if necessary.

The added chitosan has excellent stability for long-time storage, and does not harden the cloth. The chitosan is added to the cloth to enhance the comfort of the cloth. The chitosan will have hollow shape after reacting with acid solution to provide isolation for cloth upon combining with cloth, thus isolating the harmful and minute metal content of the cloth.

The antiseptic chitosan can also be absorbed by human and will activate human cell, thus enhancing immunity. The chitosan can also be added to fiber to provide sweat-absorbing, waterproof, antiseptic, deodorization.

To sum up, the protective film formed by spraying chitosan according to the present invention has the advantages of decreasing friction and reducing disease.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

I claim

1. An absorbing article for skin care and antisepsis, comprising
a substrate having absorbing ability for liquid and powder;
an augmentation layer made of chitosan and added to the substrate.

2. The absorbing article for skin care and antisepsis as in claim 1, wherein the augmentation layer is made of powder, particle, liquid or solution of chitosan.

3. The absorbing article for skin care and antisepsis as in claim 2, wherein the content of chitosan in the solution is 0.05%-15% and the pH value of the solution is adjusted to between 1.0-7.5.

4. The absorbing article for skin care and antisepsis as in claim 1, wherein the augmentation layer is added to surface or inner part of the substrate, thus forming a protective layer on the substrate.
